(19) United States
(12) Patent Application Publication
(10) Pub. No.: US 2015/0351658 A1
(43) Pub. Date: Dec. 10, 2015

(54) MEDICAL DEVICE FOR IMAGING AND METHOD FOR OPERATION THEREOF

(71) Applicant: Siemens Aktiengesellschaft, Muenchen (DE)

(72) Inventor: Andreas Kiepfer, Erlangen (DE)

(73) Assignee: Siemens Aktiengesellschaft, Muenchen (DE)

(21) Appl. No.: 14/735,567

(22) Filed: Jun. 10, 2015

(30) Foreign Application Priority Data
Jun. 10, 2014 (DE) 102014211034.3

(51) Int. Cl. A61B 5/055; A61B 5/00

(52) U.S. Cl. CPC A61B 5/055; A61B 5/742

(57) ABSTRACT

A medical device has an image generation module to generate an image of a subject, an adjustment module to adjust at least one parameter that is used by the image generation module to generate the image, and an evaluation module to evaluate whether the image generated by the image generation module based on the adjustment of the adjustment module, deviates by a predefined amount from a predefined set value. An access module accesses a memory in which units are stored, with which a specific running sequence for the generation of an image can be run by the image generation module. The access module accesses the memory to download a unit if the evaluation by the evaluation module shows that the image generated by the image generation module on the basis of the adjustment of the adjustment module deviates more than by the predefined amount from a predefined set value.

### BACKGROUND OF THE INVENTION

1. Field of the Invention

The present invention is in the fields of medical imaging devices and information technology.

2. Description of the Prior Art

In medical devices for imaging, such as, for example, magnetic resonance tomography (MRT) apparatuses, computed tomography (CT) apparatuses, X-ray devices, ultrasound devices, a tomosynthesis devices, etc., there are a number of adjustment options and enhancements that result in an ideal imaging method. This knowledge (know-how) is often held by a manufacturer of the medical device for imaging and likewise by external research institutions and practices.

It is extremely difficult, with a magnetic resonance tomography apparatus in particular, to find ideal procedures for acquiring an image, with which a patient can receive an optimum diagnosis. There are a number of parameters that the staff operating the magnetic resonance tomography apparatus has to determine in order to find the ideal procedure for the ideal image for diagnosis. The ideal procedure includes, for example, an ideal program sequence for a computer program that controls the magnetic resonance tomography scanner, ideal pauses, ideal injection(s) and ideal times for specific medical diagnoses.

This makes working with the magnetic resonance tomography apparatus, and with medical devices for imaging in general, extremely difficult and time-consuming for the staff operating such systems. As a result, the use of a medical device is rendered more expensive so that in some cases it may possibly fail to be used for diagnosis for cost-saving reasons alone.

### SUMMARY OF THE INVENTION

An object of the present invention is to provide a medical device for imaging and a method for a medical device for imaging that solve the aforementioned problems.

This object is achieved in accordance with the invention by a medical device for imaging that has an image generation module for the generation of an image, an adjustment module to adjust at least one parameter that is used by the image generation module for the generation of an image. An evaluation module evaluates whether the image generated by the image generation module on the basis of the adjustment of the adjustment module deviates by a predefined amount from a predefined set value. An access module accesses a memory in which units are stored, with which units a specific running sequence for the generation of an image by the image generation module can be implemented. The access module for accessing the memory is designed to download a unit if an evaluation by the evaluation module shows that the image generated by the image generation module on the basis of the adjustment of the adjustment module deviates by more than the predefined amount from a predefined set value, and/or the access module for accessing the memory is designed to download a unit if an evaluation by the evaluation module shows that the image generated by the image generation module on the basis of the adjustment of the adjustment module deviates less than by the predefined amount from a predefined set value. As an alternative to the evaluation module, access to the procedures in the memory can also be open to any user of a device (without further checks and comparisons with set reference values). In this case the memory is simply used; there is no level that has to be reached nor any automatic system.

With the medical device it is possible to incorporate specific running sequences for imaging retrospectively in the medical device as required such that they can be run without an installation team from the manufacturer being needed for this. The sequences are therefore incorporated automatically.

In addition, it is possible for the team operating the unit to use available know-how from another hospital in diagnosis, for example, of the liver, of the knee, and so on, without losing or expending a substantial amount of time. This means that outstanding images can be delivered by the medical device in a fast, simple, and therefore economical manner so that the medical diagnosis is simplified considerably.

Furthermore, it is possible for the operator of the medical device, to pass on know-how acquired when working with the medical device to other hospitals or research institutions, in a straightforward manner.

The at least one parameter in the medical device can be selected from the group consisting of: a process in the acquisition sequence for the acquisition of an image, a pause in the running of the acquisition sequence, an injection to be administered to the body that is to be investigated, a time for specific medical diagnoses.

It is possible for the unit to be a computer program in a specific running sequence for imaging, a computer program update and/or hardware.

In one variant, the access module can be designed, for example, to always access the memory to upload a specific running sequence if the running sequence-preparation module has prepared a specific running sequence.

The medical device can additionally have an installation module to install a specific running sequence for the generation of an image by the image generation module, which sequence was downloaded by the access module in the memory.

The medical device can additionally have a display module to display data for operating the medical device, the access module in the memory being designed to request a code on the display module when the access module accesses the memory.

In a further variant, the access module is designed to access an external memory, in which the computer programs can be stored.

The aforementioned object also is solved by a method for a medical device for imaging in accordance with the invention that includes the steps of adjusting, with an adjustment module, at least one parameter that is used by the image generation module to generate an image, generating an image, with an image generation module, evaluating, with an evaluation module, whether the image generated by the image generation module on the basis of the adjustment of the adjustment module deviates from a predefined set value by a predefined amount. The method also includes accessing, with an access module, a memory in which units can be stored, with which a specific running sequence to generate an image can be executed by the image generation module. The access module accesses the memory to download a unit if an evaluation by the evaluation module shows that the image generated by the image generation module on the basis of the adjustment of the adjustment module deviates from a predefined set value more than by the predefined amount, and/or the access module accesses the memory to upload a specific running sequence if an evaluation by the evaluation module shows that the image generated by the image generation module on the basis of the adjustment of the adjustment module deviates from a predefined set value less than by the predefined amount.

The method has the same advantages as described above for the medical device.

The present invention also encompasses a non-transitory, computer-readable data storage medium encoded with programming instructions, the storage medium being loaded into a processor, or distributed among a number of processors, of a medical imaging device, and the programming instructions causing the processor or processors to then execute the method according to the invention as described above.

Within the scope of the invention, not all the steps in the method necessarily have to be carried out on one and the same computer entity. The steps can also be carried out on different devices or entities. It is also possible for individual segments of the method described in the aforementioned to be carried out in one commercial unit and the remaining components in a different commercial unit, as a distributed system. Optionally, it is also possible for the sequence of process steps to be varied.

It is described hereinafter in even more detail how the problem is solved both with reference to the claimed medical device and also to the claimed method. Features, advantages or alternative embodiment likewise also be transferred to the other aspects, and vice versa. The medical device can be further developed with the features described in connection with the method, and vice versa. The corresponding functional features of the method are formed by corresponding apparatus modules, in particular by hardware modules.

### BRIEF DESCRIPTION OF THE DRAWINGS

FIG. 1 is an overview of modules according to a preferred embodiment of the invention.

FIG. 2 is a data flow diagram for the method according to a preferred embodiment of the invention.

FIG. 3 is an overview of modules according to a second preferred embodiment of the invention.

### DESCRIPTION OF THE PREFERRED EMBODIMENTS

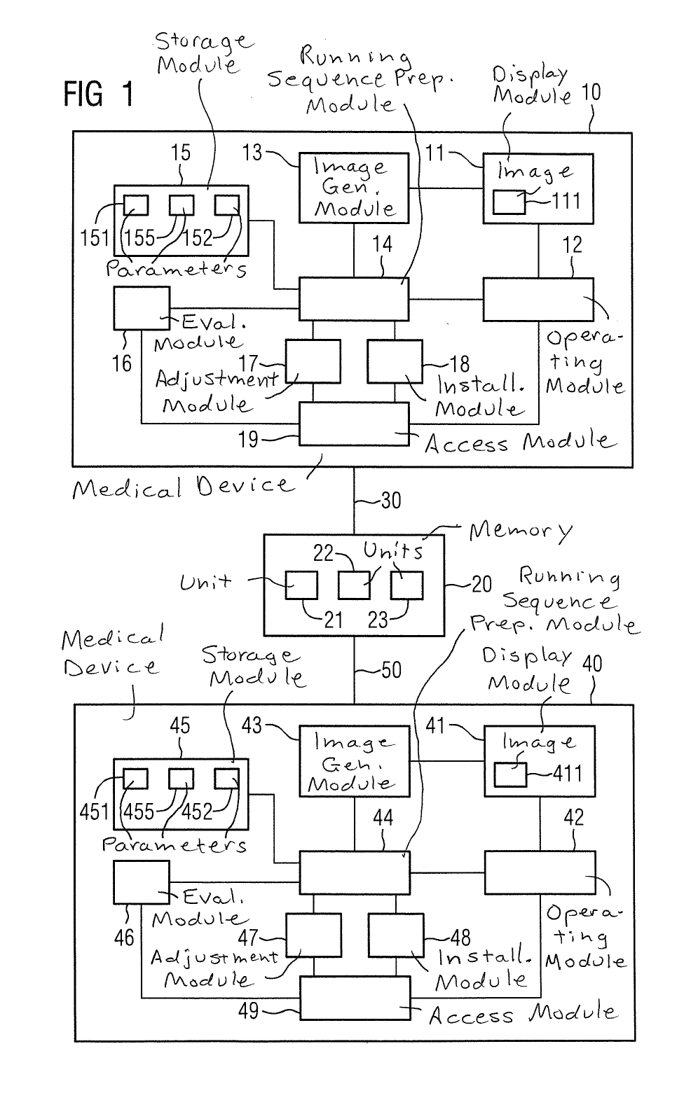

FIG. 1 shows a first medical device 10 and an external memory 20, which are connected to each other via a first connection cable 30, and a second medical device 40, which is likewise connected to the external memory 20 by a second connection cable 50. The connection cable(s) 30, 50 can be part of a data network, in particular a local network (LAN), a wide area network (WAN), the internet, etc. The connection cable(s) 30, 50 can be wired or wireless. The connection cable(s) 30, 50 can also be designed as lightwave conductors.

The first medical device 10 has a display module (module) 11 to display an image 111, an operating module 12, an image generation module (scanner) 13, a running sequence-preparation module 14, a storage module (memory) 15 to store multiple parameters 151, 152 and a predefined set value for the image 111, an evaluation module 16, an adjustment module 17, an installation module 18 and an access module 19. Each of these modules can be a processor, or formed as parts of one or more shared processors.

In the memory 20, units 21, 22, 23 are stored and/or further units, which are not shown and which may be used during the operation of the medical device 10, can be stored. In this embodiment, the unit 21 is a computer program, with which a specific running sequence for imaging can be run on the medical device 10. In this embodiment, the unit 22 is a software-update, with which a computer program or operating system installed on the medical device 10 can be updated. In this embodiment, the unit 23 is a specification for a hardware component, which a user or operator of the medical device 10 can obtain and can additionally install on their medical device 10 the respective hardware component after purchase thereof individually and/or with the use of the manufacturer of the medical device 10.

Alternatively or additionally, a manufacturer and/or service provider may also upload or offer to supply updates/running sequences without medical devices, although this is not shown in FIG. 1. Uploading and offering mostly ensues in this case in the use of modules 14, 17, 18, 19, in which no medical device is required.

The second medical device 40 has a display module (monitor) 41 to display an image 411, an operating module 42, an image generation module (scanner) 43, a running sequence-preparation module 44, a storage module 45 to store multiple parameters 451, 452 and a predefined set value 455 for the image 411, an evaluation module 46, an adjustment module 47, an installation module 48 and an access module 49. The second medical device 40 is designed in the same way as the first medical device 10, so that reference is made to the description of the first medical device 10 for the description of the modules in the second medical device 40 and the functions thereof.

The first medical device 10 and the external memory 20 can exchange data via the first connection cable 30 with the aid of the access module 19. In addition, the second medical device 40 and the external memory 20 can exchange data via the second connection cable 50 with the use of the access module 49. Here, each of the access modules 19, 49, for example, can upload a unit 21 into the memory 20, so that the unit 21 can be stored in the memory 20. In addition, the access modules 19, 49 can each download one or a number of the units 21, 22, 23 from the memory 20. The corresponding unit 21, 22, 23 can then be installed on the corresponding medical device 10, 40 with the use of the corresponding installation module 18, 48 and thus become executable on the corresponding medical device 10, 40 such that the unit can be used in imaging with the corresponding medical device 10, 40.

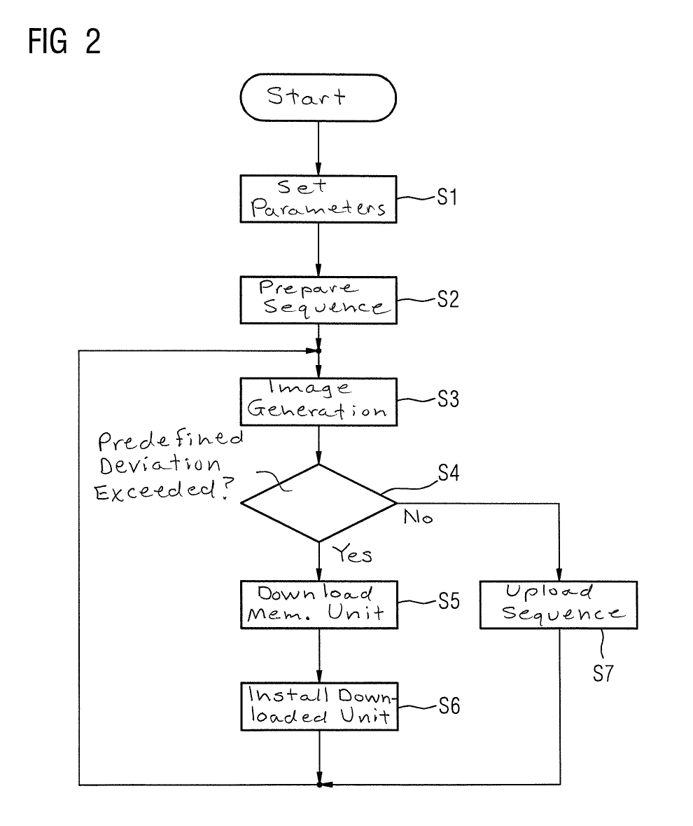

The function of the medical devices 10, 40 is described in even greater detail with the use of FIG. 2 together with the method carried out by the medical device 10. The medical device 40 can carry out the same method, although this is not described again here.

According to FIG. 2, after the method has begun, in a step S1, the adjustment module 17 of the medical device 10 sets one or more parameters 151, 152 on the basis of a basic setting and/or corresponding inputs from a user on an operating module 12 which are to be used to generate an image with the image generation module 13. The parameters 151, 152 are for example, a procedure in an acquisition sequence for acquiring the image 111 of a human or animal body, a pause in the procedure for the acquisition sequence, an injection to be administered to the human or animal body that is to be diagnosed, a time for specific medical diagnoses, etc. The flow sequence subsequently proceeds to a step S2.

In step S2, the running sequence-preparation module 14 prepares a running sequence for imaging on the basis of the parameter or parameters 151, 152 set in step S1. The flow sequence subsequently proceeds to a step S3.

In step S3, the image generation module 13 generates an image 111, which is displayed on the display module 11 for a user of the medical device 10. Depending on the quality of the image 111, the image 111 can already be used for diagnosis or not. The flow sequence subsequently proceeds to a step S4.

In step S4, the evaluation module 16 evaluates whether the image 111 corresponds to the predefined set value 155 or not. This predefined set value 155 can be a reference image stored beforehand and/or details relating to contrast and/or include brightness and/or image definition and so on. If the image 111 generated by the image generation module 13 deviates from the predefined set value 155 by more than by the predefined amount, the flow sequence proceeds to a step S5. If, however, there is no or less deviation present, the flow sequence subsequently proceeds to a step S7. As an alternative to step S4, access to the procedures running in the memory 20 can also be open to any user of a random device. In this case, step S4 is omitted and the memory 20 is simply used by the random device, there is no level that has to be achieved nor any automatic mechanism.

In step S5 the access module 19 accesses the external memory 20 and downloads, for example, the unit 21, which promises better results in image generation with the image generation module 13. It is possible for an access code for the memory 20, such as a user name and/or a password or biometric data, a barcode, etc. to be requested from the user on the operating module 12 and/or on the display module 11. In addition, it is possible for the user to likewise enter details relating to specific conditions such as payment method, license, and so on in order to use the unit 21. The flow sequence subsequently proceeds to a step S6.

In step S6, the installation module 18 installs the unit 21 downloaded by the memory 20, for example, into the storage module 15 or 45, so that the unit 21 can be used for image generation with the image generation module 13. The flow sequence subsequently reverts to a step S3.

In step S7, the access module accesses the memory 20 in order to upload the specific running sequence that has just been generated into the external memory 20. Here, it is also possible beforehand with the use of the operating module 12 and/or the display module 11, for a query to be sent to the user as to whether the specific running sequence is to be uploaded as unit 21 onto the external memory 20 or not. It is likewise possible here for an access code for the memory 20, such as a user name and/or a password or biometric data, a barcode, and so on to be requested from the user. In addition, it is possible for the user to likewise input details relating to specific conditions such as price, license, and so on when using the unit 21. The flow sequence subsequently reverts to a step S3.

As required, the method can, for example, revert to step S1 again through an input on the operating module 12.

The method is terminated when the medical device 10 is switched off.

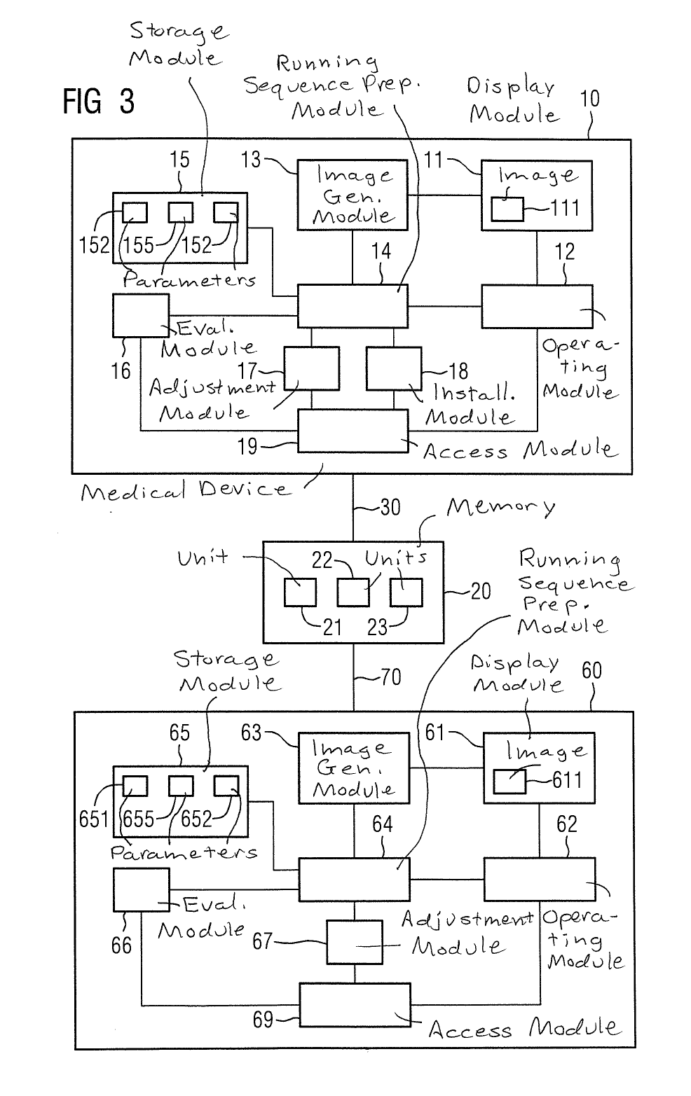

FIG. 3 shows a second embodiment, according to which the medical device 10 and a third medical device 60 are connected to the memory 20 via connection cables 30, 70. The connection cable 70 can be designed according to one of the design variants of the connection cable 30 as described in the aforementioned.

The third medical device 60 has a display module (monitor) 61 to display an image 611, an operating module 62, an image generation module 63, a running sequence-preparation module 64, a storage module 65 to store a plurality of parameters 651, 652 and a predefined set value 655 for the image 611, an evaluation module 66, an adjustment module 67, an installation module 68 and an access module 69. The third medical device 60 is broadly constructed in the same way as the first medical device 10, so that reference is made to the description of the first medical device 10 for the description of the modules in the third medical device 60 and the functions thereof.

Unlike the first medical device 10, the medical device 60 does not have an installation module 18 to install one of the units 21, 22, 23. Consequently, the medical device 60 can only upload specific running sequences as unit 21 to the external memory 20, whereas the medical device 10 is designed as described with reference to the first embodiment.

In the embodiments described in the aforementioned, it is also possible for more than two medical devices 10, 40, 60 to be connected to the memory 20 via one connection cable 30, 50, 70. In addition, the number and type of units 21, 22, 23 are selectable at random in the memory 20.

Moreover, it is possible in the embodiments described in the aforementioned for whole investigation sequences to be prepared from a number of running sequences and from specific parameterization, these sequences then being uploaded as unit 21 into the memory 20.

Although modifications and changes may be suggested by those skilled in the art, it is the intention of the inventor to embody within the patent warranted hereon all changes and modifications as reasonably and properly come within the scope of his contribution to the art.

I claim as my invention:

1. A medical device for imaging, comprising:
an image generation module to generate an image;
an adjustment module to adjust at least one parameter that is used by the image generation module to generate an image;
an evaluation module to evaluate whether the image generated by the image generation module on the basis of the adjustment of the adjustment module deviates by a predefined amount from a predefined set value;
an access module for accessing a memory, in which units with which a specific running sequence for the generation of an image can be run by the image generation module are storable;
wherein the access module for accessing the memory is designed to download a unit if an evaluation by the evaluation module shows that the image generated by the image generation module on the basis of the adjustment of the adjustment module deviates by more than the predefined amount from a predefined set value; and
wherein the access module for accessing the memory is designed to upload a specific running sequence if an evaluation by the evaluation module shows that the image generated by the image generation module on the basis of the adjustment of the adjustment module deviates by less than the predefined amount from the predefined set value.

2. The medical device as claimed in claim 1, wherein the at least one parameter can be selected from the group consisting of: a process in the acquisition sequence for the acquisition of the image, a pause in the running of the acquisition sequence, an injection to be administered, a time for specific medical diagnoses.

3. The medical device as claimed in claim 1, wherein the unit is a computer program in a specific running sequence for imaging, a computer program update or hardware.

4. The medical device as claimed in claim 1, wherein the access module is designed to access the memory to upload a specific running sequence if a running sequence-preparation module has prepared a specific running sequence.

5. The medical device as claimed in claim 1, which is additionally designed with an installation module to install a specific running sequence, downloaded by the access module in the memory, to generate an image with the image generation module.

6. The medical device as claimed in claim 1, which is additionally designed with a display module to display data for operating the medical device, the access module being designed to request an access code on the display module when the access module accesses the memory.

7. The medical device as claimed in claim 1, wherein the access module is designed to access an external memory in which the units can be stored.

8. A method for operating a medical imaging device, comprising:
adjusting, with an adjustment module, at least one parameter, which is used by the image generation module to generate an image;
generating, with an image generation module, an image, which can be used for diagnosis on the human and/or animal body;
evaluating, with an evaluation module, whether the image generated by the image generation module on the basis of the adjustment of the adjustment module deviates from a predefined set value by a predefined amount;
accessing, with an access module, a memory, in which units can be stored, with which a specific running sequence to generate an image can be run by the image generation module;
wherein the access module accesses the memory to download a specific running sequence if an evaluation by the evaluation module shows that the image generated by the image generation module on the basis of the adjustment of the adjustment module deviates from the predefined set value more than by the predefined factor; and
wherein the access module accesses the memory to upload a specific running sequence if an evaluation by the evaluation module shows that the image generated by the image generation module on the basis of the adjustment of the adjustment module deviates from the predefined set value less than by the predefined factor.

9. A non-transitory, computer-readable data storage medium encoded with programming instructions, said storage medium being loaded into at least one processor of a medical imaging device, and said storage medium being encoded with programming instructions that cause said at least one processor to:
adjust, with an adjustment module, at least one parameter, which is used by the image generation module to generate an image;
generate, with an image generation module, an image, which can be used for diagnosis on the human and/or animal body;
evaluate, with an evaluation module, whether the image generated by the image generation module on the basis of the adjustment of the adjustment module deviates from a predefined set value by a predefined amount;
access, with an access module, a memory, in which units can be stored, with which a specific running sequence to generate an image can be run by the image generation module;
cause the access module to access the memory to download a specific running sequence if an evaluation by the evaluation module shows that the image generated by the image generation module on the basis of the adjustment of the adjustment module deviates from the predefined set value more than by the predefined factor; and
cause the access module to access the memory to upload a specific running sequence if an evaluation by the evaluation module shows that the image generated by the image generation module on the basis of the adjustment of the adjustment module deviates from the predefined set value less than by the predefined factor.
